(19) United States
(12) Patent Application Publication
(10) Pub. No.: US 2026/0166571 A1
(43) Pub. Date: Jun. 18, 2026

(54) GASLESS SPRAY TIP FOR VISCOUS FLUIDS

(71) Applicants: Baxter International Inc., Deerfield, IL (US); Baxter Healthcare SA, Glattpark (Opfikon) (CH)

(72) Inventor: Christopher Moss, Wheeling, IL (US)

(21) Appl. No.: 19/425,489

(22) Filed: Dec. 18, 2025

Related U.S. Application Data
(60) Provisional application No. 63/735,734, filed on Dec. 18, 2024.

(51) Int. Cl. B05B 1/34; A61B 17/00

(52) U.S. Cl. CPC B05B 1/341; A61B 17/00491; A61B 2017/00495; A61B 2017/00522

(57) ABSTRACT

A spray tip assembly for providing a viscous fluid includes a body comprising an outlet orifice; and an insert configured to fit within the body. The spray tip assembly comprises a plurality of channels tangent to and in fluid communication with a hollow chamber. Each of the plurality of channels has a length between about 0.4 mm and about 1.0 mm.

### CROSS-REFERENCE TO RELATED APPLICATIONS

The present application claims the benefit of U.S. Provisional Application No. 63/735,734, entitled GASLESS SPRAY TIP FOR VISCOUS FLUIDS and filed Dec. 18, 2024, the contents of which are hereby incorporated by reference in their entirety.

### BACKGROUND

High viscous fluids, such as hemostats, sealants, and adhesion barriers may be applied to surgical sites using spray systems. The spray systems can include gas-assisted spray systems. However, these systems are expensive to maintain and may require long set-up times leading to delay in surgery. Further, gas-assisted systems require a battery and gas supply. Complication during surgery may arise in the event the batteries are dead or there is a depleted gas supply.

Other systems for administering high viscous fluids include pressure swirl atomizers. However, existing pressure swirl atomizers suffer from performance issues. For example, pressure swirl atomizers may not be well suited for high viscous fluids that require low spray pressures at low flow rates.

There remains a need for improved gasless spray systems, such as pressure swirl atomizers, that are suited for delivering high viscous fluids, such as hemostats, sealants, and adhesion barriers at low flow rates and low pressure.

### SUMMARY

In light of the disclosure herein, and without limiting the scope of the invention in any way, a first aspect of the present disclosure, which may be combined with any other aspect listed herein unless specified otherwise, provides a spray tip assembly for providing a viscous fluid, the spray tip assembly comprising a body comprising an outlet orifice; and an insert configured to fit within the body. The spray tip assembly comprises a plurality of channels tangent to and in fluid communication with a hollow chamber, and the channels have a length between about 0.4 mm and about 1.0 mm.

According to a second aspect of the present disclosure, which may be combined with any other aspect listed herein, a diameter of the hollow chamber is between about 1.6 mm to about 2.2 mm.

According to a third aspect of the present disclosure, which may be combined with any other aspect listed herein, the diameter of the hollow chamber is about 1.8 mm.

According to a fourth aspect of the present disclosure, which may be combined with any other aspect listed herein, the plurality of channels comprises two channels.

According to a fifth aspect of the present disclosure, which may be combined with any other aspect listed herein, the outlet orifice is concentric with and smaller than the hollow chamber.

According to a sixth aspect of the present disclosure, which may be combined with any other aspect listed herein, a width of each of the plurality of channels is between about 0.1 mm and 0.3 mm.

According to a seventh aspect of the present disclosure, which may be combined with any other aspect listed herein, the width of each of the plurality of channels is about 0.2 mm.

According to an eighth aspect of the present disclosure, which may be combined with any other aspect listed herein, each of the plurality of channels comprises a square cross-section.

According to a ninth aspect of the present disclosure, which may be combined with any other aspect listed herein, the body comprises the hollow chamber and the plurality of channels.

According to a tenth aspect of the present disclosure, which may be combined with any other aspect listed herein, the insert comprises the hollow chamber and the plurality of channels.

According to an eleventh aspect of the present disclosure, which may be combined with any other aspect listed herein, the length of each of the plurality of channels is 0.6 mm.

According to a twelfth aspect of the present disclosure, which may be combined with any other aspect listed herein, an outside diameter of the body is less than 5.5 mm.

According to a thirteenth aspect of the present disclosure, which may be combined with any other aspect listed herein, the body comprises a cylindrical chamber for fitting the insert, and wherein the cylindrical chamber has a diameter of between 3 mm and about 4 mm.

According to a fourteenth aspect of the present disclosure, which may be combined with any other aspect listed herein, the hollow chamber has a height of between about 0.1 mm to about 0.3 mm.

According to a fifteenth aspect of the present disclosure, which may be combined with any other aspect listed herein, the outlet orifice has a diameter of between about 0.2 mm and 0.3 mm.

According to a sixteenth aspect of the present disclosure, which may be combined with any other aspect listed herein, the outlet orifice has a length of between about 0.1 mm and 0.4 mm.

According to a seventeenth aspect of the present disclosure, which may be combined with any other aspect listed herein, the insert comprises a plurality of protrusions surrounding at least a portion of the outside surface of the insert.

According to an eighteenth aspect of the present disclosure, which may be combined with any other aspect listed herein, the insert comprises a proximal end and a distal end, wherein the proximal end comprising a flat surface and the proximal end comprising the hollow chamber and the plurality of feeders.

According to a nineteenth aspect of the present disclosure, which may be combined with any other aspect listed herein, a diameter of the insert is between about 2.6 mm and 3.6 mm.

According to a twentieth aspect of the present disclosure, which may be combined with any other aspect listed herein, the spray tip assembly is configured to deliver the viscous fluid at a flow rate of between about 30 ml/min and about 60 ml/min.

According to a twenty first aspect of the present disclosure, which may be combined with any other aspect listed herein, a diameter of the hollow chamber is between about 0.6 mm to about 0.8 mm.

According to a twenty second aspect of the present disclosure, which may be combined with any other aspect listed herein, a spray tip for mixing and dispensing a viscous fluid is provided. The spray tip comprises a swirl chamber at an end portion of the spray tip, the swirl chamber comprising: a plurality of channels tangent and in fluid communication to a hollow chamber, wherein the length of the plurality of channels is about 0.6 mm or less and the diameter of the hollow chamber is about 1.8 mm or less. The swirl chamber is configured to impart rotation to the viscous fluid introduced in the spray tip.

According to a twenty third aspect of the present disclosure, which may be combined with any other aspect listed herein, a spray system comprises a multi-component fluid; and a spray tip for mixing components to form the multi-component fluid and dispensing the multi-component fluid. The spray tip comprises a swirl chamber at an end portion of the spray tip, the swirl chamber comprising a plurality of channels tangent and in fluid communication to a hollow chamber, wherein the length of the plurality of channels is about 0.6 mm or less and the diameter of the hollow chamber is about 1.8 mm or less. The swirl chamber is configured to impart rotation to the multi-component fluid after being mixed within the spray tip.

According to a twenty fourth aspect of the present disclosure, which may be combined with any other aspect listed herein, the multi-component fluid comprises a surgical sealant.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE DRAWINGS

FIG. 1A illustrates a perspective view of a spray tip body of a spray tip assembly according to an example of the present disclosure.

FIG. 1B illustrates a cross-sectional view of a spray tip body of a spray tip assembly according to an example of the present disclosure.

FIG. 1C illustrates a front view of an end of a spray tip body of a spray tip assembly according to an example of the present disclosure.

FIG. 2A illustrates a swirl chamber of a spray tip assembly according to an example of the present disclosure.

FIGS. 2B to 2C illustrate a side view of an insert of a spray tip assembly according to an example of the present disclosure.

FIG. 3A illustrates a swirl chamber of a spray tip assembly according to another example of the present disclosure.

FIGS. 3B to 3C illustrate a side view of an insert of a spray tip assembly according to another example of the present disclosure.

FIGS. 4A to 4C are images of spray patterns resulting from a spray tip assembly of a first design with feeder length of 0.6 mm and a spin chamber diameter of 1.8 mm.

FIGS. 5A to 5C are images of spray patterns resulting from a spray tip assembly of a second design with feeder length of 1.15 mm and a spin chamber diameter of 0.7 mm.

FIGS. 6A to 6C are images of spray patterns resulting from spray tip assembly of a third design with feeder length of 0.35 mm and a spin chamber diameter of 0.6 mm.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well¬-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specific the presence of stated features, integers, steps, operations, elements, and/or components, but do not preclude the presence or additional of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

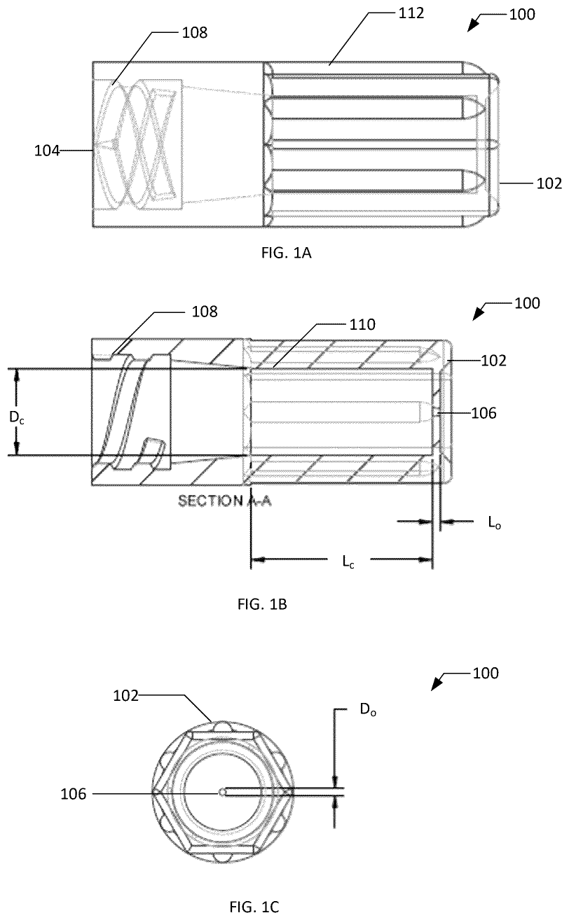

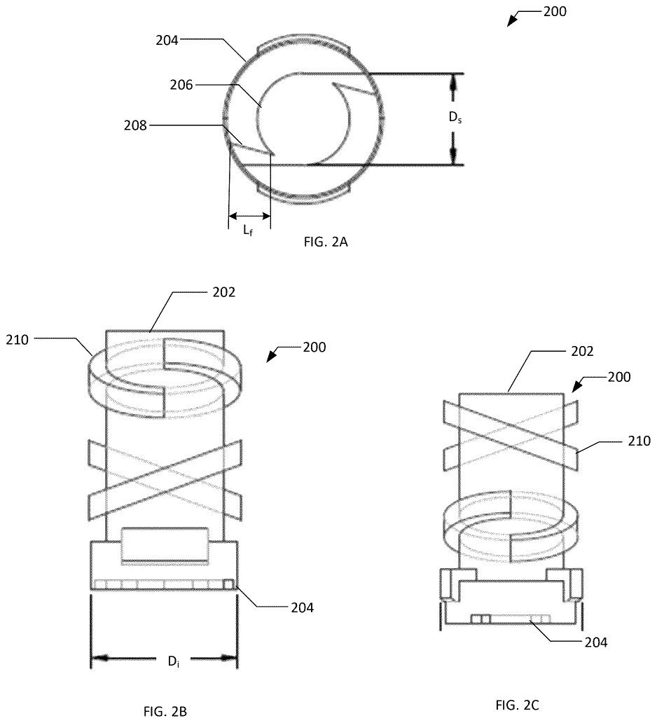

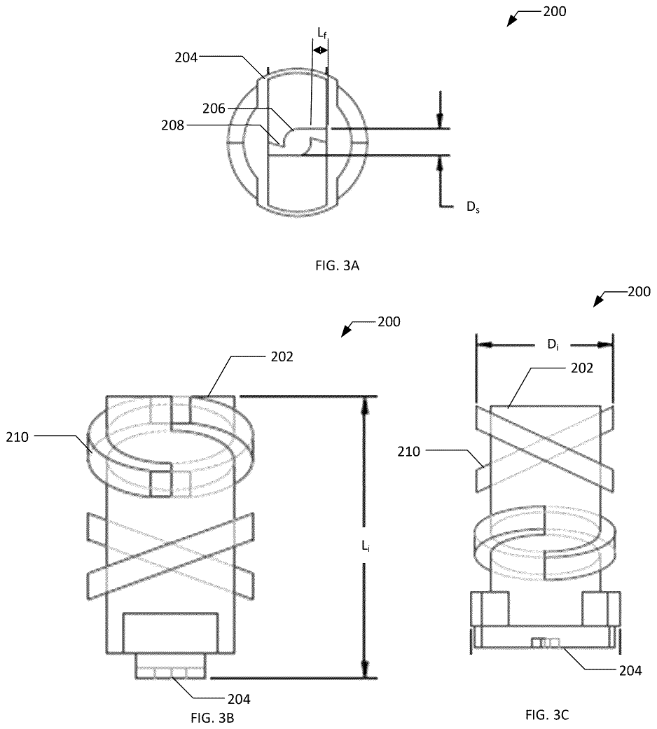

The present disclosure generally describes a gasless spray tip assembly for delivering viscous fluids at a low pressure (e.g. without the used of mechanical aid) and a low flow rate. In some embodiments, a viscous fluid has a viscosity of between about 80 cp to about 120 cp, more preferably about 100 cp. In some embodiments, a low flow rate is between about 30 ml/min and 60 ml/min. Spray tip assemblies according to the present disclosure show reliable and improved spray characteristics. Referring to the drawings, FIGS. 1A through 3C illustrate example embodiments of a spray tip assembly for delivering high viscous fluids. The spray tip assembly includes a spray tip body 100 as illustrated in FIG. 1A through 1C and an insert 200 as illustrated in FIG. 2A through 3C.

### Spray Tip Body

FIGS. 1A to 1C illustrate a spray tip body 100 according to an example of the present disclosure. As illustrated in FIGS. 1A to 1C, the spray tip body 100 may include an outlet end 102 and an attachment end 104. The spray tip body 100 also includes an outlet orifice 106 at the outlet end 102 as shown in FIG. 1C. The outlet orifice has a diameter of Dₒ and a length of Lₒ. The present inventors have determined that the outlet orifice diameter Dₒ and length Lₒ contribute to the spray properties of viscous fluids. For example, a smaller outlet orifice exhibits improved spray with lower pressure and lower flow rates. In some embodiments, the outlet orifice diameter Dₒ is about 0.26 mm and the outlet orifice length Lₒ is about 0.3 mm.

The spray tip body 100 may also include a threaded cannula portion 108 at the attachment end 104, which may facilitate removeable connection of spray tip assembly to a fluid source, such a syringe or other vessel. For example, threading or threads may threadingly engage with corresponding threading or threads of the fluid source. In another example, spray tip body 100 may include other attachment features to removably connect to or couple to the fluid source. For example, the spray tip body 100 may be press-fit and may maintain a friction fit with the fluid source. In some embodiments, the spray tip assembly may be removably attached to a larger applicator device and replaced as necessary, for example, when the fluid solidifies quickly.

To aid in the removal and attachment of the spray tip assembly with the fluid source, an outside surface of the spray tip body 100 may include a gripping portion 112. The gripping portion 112 may include ridges, protrusions, a textured surface, or other surface finish or surface geometry that aids with gripping the spray tip body 100.

The spray tip body 100 may be generally cylindrical and may include a hollow chamber 110 at the outlet end 102. The chamber 110 may be configured to receive an insert, which will be described in more detail herein. The size and shape of the chamber 110 may be optimized for improved spraying characteristics of the viscous compositions. Further, an outer diameter of the spray tip body 100 may be sized to enable access to certain areas for delivery of the composition. In some embodiments, the spray tip assembly may be used in laparoscopic procedures requiring an outer diameter of less than 5.5 mm. In some embodiments, the chamber 110 is sized depending on the specification required for the outer diameter of the spray tip body 100. For example, the chamber 110 may be sized such that the wall thickness of the spray tip body 100 is thick enough to maintain rigidity. In some embodiments, the chamber diameter De is between about 3 mm and about 4 mm, more preferably about 3.3 mm.

### Spray Tip Insert

FIGS. 2A to 3C illustrate tip inserts 200 according to examples of the present disclosure. In an example, the tip insert 200 is non-removably assembled into the spray tip body 100, such as in the chamber 110, via a press fit. In other embodiments, the insert 200 is removably coupled to the spray tip body 100. As fluid is pushed through the spray tip assembly from the fluid source, the fluid enters the chamber 110 housing the insert 200. The insert 200 forces the fluid to the outermost edge of the chamber 110 between the insert 200 and the wall of the chamber 110. As more fluid enters chamber 110, the fluid is pushed through the chamber 110 and out of the outlet orifice 106 of the spray tip body 100.

The insert 200 may be sized to fit into the chamber 110, so the size and shape of the insert 200 may depend on the required specifications of the spray tip body 100. In some embodiments, the insert 200 is generally cylindrical. The insert 200 may have a diameter Di of between about 2.6 mm and 3.6 mm, more preferably about 3.3 mm. In other embodiments, the insert 200 is any desired shape. In some embodiments, the insert 200 is partially hollow.

The insert 200 may have a proximal end 202 and a distal end 204 closest to the outlet orifice 106 when housed within the spray tip body 100. The proximal end 202 may include a blunt or flat fluid contact surface, which may be the first surface the fluid encounters. As illustrated in FIGS. 2A and 3A, the distal end 204 may include a swirl chamber portion for imparting rotation of the fluid as it exits the spray tip assembly. The swirl chamber portion may include a swirl chamber 206 and a plurality of feeders 208 leading into the swirl chamber 206. In some embodiments, the swirl chamber 206 is generally cylindrical in shape. In other embodiments, the swirl chamber 206 is generally conical or includes any suitable geometry. In some embodiments, the geometry and size of the swirl chamber 206 produces a preferred spray geometry of the viscous fluid.

As discussed, the insert 200 includes a plurality of feeders 208 tangent to the swirl chamber 206, which feed the fluid from the edge of the chamber 110 to the swirl chamber 206. In some embodiments, the spray tip assembly includes two feeders 208, three feeders 208, four feeders 208, five feeders 208, or any suitable number of feeders 208. In some embodiments, the feeders 208 have a square cross-section or a trapezoidal cross-section. In some examples, the feeders 208 and the swirl chamber 206 are molded into the distal end 204 of the tip insert 200. In other examples, the feeders 208 and the swirl chamber 206 are molded into an outlet end 102 of the spray tip body 100. The present inventors have determined that the size of the swirl chamber 206 and the feeders 208 impacts the spray characteristics of the spray tip assembly.

In some embodiments, the feeders 208 have a relatively short length and a small cross-section. The small cross section may increase initial fluid speed at a given flow rate while shorter feeders may prevent too much head loss. In some embodiments, the length of each feeder L_{f} is between 0.4 mm and 1.0 mm, more preferably about 0.6 mm. In some embodiments, the width of each feeder is between 0.1 mm and 0.3 mm, more preferably 0.2 mm.

In some embodiments, the swirl chamber may have a relatively large diameter Dₛ compared to the diameter of the insert 200 and/or a relatively short axial height. In some embodiments, the swirl chamber 206 diameter Dₛ is between about 1.6 mm to about 2.2 mm. Additionally or alternatively, in some embodiments, the height of the swirl chamber 206 is between about 0.1 mm to about 0.3 mm, most preferably about 0.2 mm.

As shown in FIG. 3A, in some embodiments, the swirl chamber 206 may have a relatively short diameter Dₛ. For example, in some embodiments, the swirl chamber diameter Dₛ is between about 0.5 mm to about 0.9 mm, between about 0.6 mm to about 0.8 mm, more preferably about 0.7 mm.

The liquid travels through the feeders 208 from the edge of the chamber 110 to the swirl chamber 206 where it spins around the swirl chamber 206 several times and travels to the center of the chamber 110. The fluid then travels out of the outlet orifice 106. In some embodiments, the outlet orifice 106 is smaller than and concentric with the swirl chamber 206. As illustrated in FIGS. 1B and 1C, the outlet orifice has a diameter Dₒ and a length Lₒ. The present inventors have also determined that adjusting the diameter and length of the orifice 106 affects the spray properties. In some embodiments, the orifice has a diameter of between about 0.2 mm and 0.3 mm, more preferably 0.26 mm, and a length of between about 0.1 mm and 0.4 mm, more preferably 0.3 mm. In another embodiment, the orifice has a diameter between 0.3 mm and 0.5 mm, more preferably about 0.4 mm.

In some embodiments, the viscous fluid sprayed is a mixture of two or more non-homogenous fluids, such as surgical hemostats, sealants, or adhesion barriers. Two-part surgical sealants are well known and have been used for a number of years. In some embodiments, the two part surgical sealants comprise a fibrinogen solution and a thrombin solution or powder or a first PEG solution and a second PEG and hydrochloric acid powder. To aid in mixing of the components before administration, the insert 200 may also include a plurality of protrusions 210 (e.g., mixing protrusions) positioned around the surface of the insert 200 upstream the swirl chamber 206 and outlet orifice 106. In the illustrated example, the insert 200 includes four sets of protrusions 210 in a staggered cross pattern. In an example, the mixing protrusions 210 may be oriented at different axial positions (e.g., 30 degrees, 45 degrees, etc.).

The protrusions 210 may be spaced to optimize the mixing of the fluid. FIGS. 2B and 2C illustrate an example protrusion 210 geometry surrounding a portion of the insert 200. As illustrated, the protrusions 210 may crisscross, respectively. Other protrusion 210 geometries may be used, for example, the posts may be helical, triangular, etc.

### EXAMPLES

### Example 1: Spray Properties of Spray Tips with Different Spin Chamber Diameters, Spin Chamber Lengths, and Number of Feeders

Multiple spray tips with differing orifice diameters, spin chamber lengths, spin chamber diameters and feeders were analyzed to determine spray coverage properties. The varying geometries of each spray tip are detailed in Table 1.

TABLE 1
Spray Tip Geometries

| | Orifice | Spin Chamber | | |
|---|---|---|---|---|
| | Diameter | Diameter | Spin Chamber | Number of |
| Sample | (mm) | (mm) | Length (mm) | Feeders |
| 1 | 0.3 | 1.8 | 1.0 | 4 |
| 2 | 0.7 | 1.8 | 1.8 | 3 |
| 3 | 0.7 | 1.0 | 1.0 | 4 |
| 4 | 0.3 | 1.8 | 1.8 | 3 |
| 5 | 0.7 | 1.8 | 1.0 | 3 |
| 6 | 0.7 | 1.8 | 1.0 | 3 |
| 7 | 0.3 | 1.0 | 1.0 | 3 |
| 8 | 0.3 | 1.0 | 1.8 | 3 |
| 9 | 0.7 | 1.8 | 1.0 | 4 |
| 10 | 0.7 | 1.8 | 1.8 | 4 |
| 11 | 0.7 | 1.8 | 1.8 | 4 |
| 12 | 0.7 | 1.8 | 1.8 | 3 |
| 13 | 0.7 | 1.0 | 1.8 | 4 |
| 14 | 0.7 | 1.0 | 1.0 | 4 |
| 15 | 0.3 | 1.0 | 1.8 | 3 |
| 16 | 0.7 | 1.0 | 1.0 | 3 |
| 17 | 0.7 | 1.8 | 1.0 | 4 |
| 18 | 0.3 | 1.8 | 1.8 | 3 |
| 19 | 0.7 | 1.8 | 1.8 | 3 |
| 20 | 0.3 | 1.0 | 1.0 | 3 |
| 21 | 0.7 | 1.8 | 1.0 | 3 |
| 22 | 0.3 | 1.8 | 1.8 | 3 |
| 23 | 0.7 | 1.8 | 1.0 | 4 |
| 24 | 0.3 | 1.8 | 1.8 | 4 |
| 25 | 0.7 | 1.0 | 1.0 | 3 |
| 26 | 0.7 | 1.0 | 1.0 | 3 |
| 27 | 0.3 | 1.0 | 1.8 | 4 |
| 28 | 0.7 | 1.0 | 1.8 | 4 |
| 29 | 0.3 | 1.0 | 1.0 | 4 |
| 30 | 0.3 | 1.0 | 1.8 | 4 |
| 31 | 0.7 | 1.0 | 1.8 | 3 |
| 32 | 0.3 | 1.0 | 1.8 | 4 |
| 33 | 0.7 | 1.8 | 1.8 | 4 |
| 34 | 0.3 | 1.8 | 1.0 | 3 |
| 35 | 0.3 | 1.0 | 1.0 | 3 |
| 36 | 0.7 | 1.0 | 1.8 | 4 |
| 37 | 0.3 | 1.8 | 1.8 | 4 |
| 38 | 0.7 | 1.0 | 1.8 | 3 |
| 39 | 0.3 | 1.0 | 1.0 | 4 |
| 40 | 0.3 | 1.8 | 1.8 | 4 |
| 41 | 0.3 | 1.0 | 1.8 | 3 |
| 42 | 0.3 | 1.8 | 1.0 | 4 |
| 43 | 0.3 | 1.0 | 1.0 | 4 |
| 44 | 0.7 | 1.0 | 1.0 | 4 |
| 45 | 0.3 | 1.8 | 1.0 | 3 |
| 46 | 0.3 | 1.8 | 1.0 | 4 |
| 47 | 0.7 | 1.0 | 1.8 | 3 |
| 48 | 0.3 | 1.8 | 1.0 | 3 |

High viscous fluids were prepared with a small amount of gel food coloring and transferred to a syringe. Each spray tip was attached to a fluid syringe for testing. The fluid was sprayed through the spray tip. The spray droplet fineness was measured according to metrics in Table 6, with 5 being a desired spray fineness and 0 being an undesirable spray fineness. Spray angle and spray area diameter were also measured. The results of the test are shown in Table 2.

TABLE 2
Results of Spray Tip Test

| | Spray | | | Spray | |
|---|---|---|---|---|---|
| | Droplet | Time to | Spray Area | Angle | |
| Sample | Fineness | Spray (sec) | Diameter (mm) | (degrees) | Comments |
| 1 | 3 | 5.46 | 80.27 | 15.895 | Spray was not |
| | | | | | consistent. Some |
| | | | | | of the time it was a |
| | | | | | spray, other times |
| | | | | | is was a stream. |
| | | | | | Ellipse shaped |
| | | | | | spray patter, not |
| | | | | | round. |
| 2 | 3 | 2.16 | 77.32 | 20.467 | N/A |
| 3 | 3 | 1.57 | 91.98 | 19.178 | Elliptical spray |
| | | | | | pattern. Test was |
| | | | | | interrupted by |
| | | | | | syringe plunger |
| | | | | | failure. |
| 4 | 0 | 6.47 | 47.58 | 0.739 | N/A |
| 5 | 3 | 2.88 | 81.64 | 28.561 | Very round spray |
| 6 | 3 | 2.78 | 82.95 | 31.191 | N/A |
| 7 | 0 | 6.81 | 49.55 | 1.327 | N/A |
| 8 | 1 | 6.57 | 47.89 | 4.481 | N/A |
| 9 | 0 | 2.12 | 59.89 | 1.193 | N/A |
| 10 | 2 | 2.85 | 89.34 | 44.759 | N/A |
| 11 | 2 | 2.84 | 90.34 | 42.488 | Course droplets in |
| | | | | | the middle of the |
| | | | | | spray, fine droplets |
| | | | | | toward the sides. |
| 12 | 3 | 2.73 | 88.85 | 37.325 | Cone angle varies |
| | | | | | throughout test |
| 13 | 3 | 1.55 | 90.92 | 29.674 | Elliptical spray |
| | | | | | pattern. Camera |
| | | | | | was started slightly |
| | | | | | later. The widest |
| | | | | | point of the spray |
| | | | | | was not |
| | | | | | perpendicular to |
| | | | | | the camera. |
| 14 | 3 | 2.40 | 88.85 | 37.325 | Elliptical spray |
| | | | | | pattern |
| 15 | 0 | 9.60 | 48.55 | 5.039 | Intermittantly |
| | | | | | stopped during |
| | | | | | spray |
| 16 | 3 | 2.69 | 77.69 | 23.132 | N/A |
| 17 | 3 | 2.66 | 75.54 | 24.586 | N/A |
| 18 | 0 | 6.71 | 49.73 | 1.174 | N/A |
| 19 | 4 | 2.28 | 93.49 | 38.138 | N/A |
| 20 | 0 | 7.01 | 32.60 | 0.970 | N/A |
| 21 | 0 | 2.29 | 53.90 | 1.825 | N/A |
| 22 | 4 | 7.56 | 65.36 | 20.759 | Sprayed |
| | | | | | intermittently, was |
| | | | | | sometimes a |
| | | | | | stream. Very fine |
| | | | | | spray in a narrow |
| | | | | | cone when it did |
| | | | | | spray. Elliptical |
| | | | | | spray pattern. |
| 23 | 0 | 2.82 | 50.92 | 1.563 | N/A |
| 24 | 0 | 8.26 | 52.59 | 0.769 | N/A |
| 25 | 3 | 2.53 | 83.50 | 26.380 | N/A |
| 26 | 3 | 2.60 | 78.54 | 19.310 | N/A |
| 27 | 0 | 7.05 | 46.28 | 0.720 | N/A |
| 28 | 4 | 1.93 | 75.56 | 29.701 | N/A |
| 29 | 0 | 8.42 | 42.45 | 2.517 | N/A |
| 30 | 0 | 7.05 | 44.21 | 1.083 | N/A |
| 31 | 2.5 | 2.30 | 101.78 | 44.010 | Spray pattern was |
| | | | | | strangely shaped |
| | | | | | (amorphous). |
| 32 | 0 | 6.75 | 47.28 | 1.199 | Intermittent flow |
| | | | | | of sealant |
| 33 | 0 | 2.41 | 55.92 | 1.436 | N/A |
| 34 | 4 | 7.40 | 83.11 | 40.977 | Started as a stream. |
| | | | | | Took some time to |
| | | | | | evolve into a spray. |
| | | | | | Elliptical spray |
| | | | | | pattern |
| 35 | 4 | 7.06 | 74.09 | 15.265 | Started as a stream. |
| | | | | | Took some time to |
| | | | | | evolve into a spray. |
| | | | | | Elliptical spray |
| | | | | | pattern |
| 36 | 2 | 2.27 | 87.33 | 31.030 | Elliptical spray |
| | | | | | pattern |
| 37 | 1 | 6.36 | 61.55 | 5.254 | Elliptical spray |
| | | | | | pattern |
| 38 | 2.5 | 2.47 | 113.07 | 49.560 | Amorphous spray |
| | | | | | pattern |
| 39 | 3 | 8.84 | 59.54 | 10.015 | Stopped |
| | | | | | intermittantly |
| | | | | | during spraying. |
| | | | | | Was sometimes a |
| | | | | | spray and |
| | | | | | sometimes a |
| | | | | | stream |
| 40 | 1 | 8.09 | 58.28 | 5.813 | Elliptical spray |
| | | | | | pattern |
| 41 | 0 | 7.73 | 50.13 | 3.681 | Stopped |
| | | | | | intermittently |
| 42 | 3 | 6.31 | 61.24 | 15.072 | Seemed to have a |
| | | | | | stream of course |
| | | | | | droplets |
| | | | | | surrounded by a |
| | | | | | spray of fine |
| | | | | | droplets |
| 43 | 4 | 17.80 | 85.41 | 29.903 | Stopped |
| | | | | | intermittently. |
| | | | | | Amorphous spray |
| | | | | | pattern |
| 44 | 3 | 2.36 | 86.61 | 25.680 | N/A |
| 45 | 4 | 6.29 | 68.63 | 31.596 | Started as a stream, |
| | | | | | took some time to |
| | | | | | evolve into a spray. |
| 46 | 4 | 7.62 | 69.27 | 22.272 | Elliptical spray |
| | | | | | pattern. Had a |
| | | | | | central core with |
| | | | | | fine mist around it. |
| 47 | 2 | 2.29 | 98.57 | 32.816 | Triangular spray |
| | | | | | pattern. |
| 48 | 4 | 6.85 | 73.54 | 19.686 | Started as a stream, |
| | | | | | took some time to |
| | | | | | evolve into a spray. |

The results indicate that spray tip samples with a smaller orifice (0.3 mm), larger swirl chamber diameter (1.8 mm), and shorter swirl chamber length (1.0 mm) tend to exhibit better spray characteristics. Small orifices in particular are desirable as the spray was generated at a lower flow rate.

### Example 2: Spray Properties of Spray Tips with Different Feeder Lengths and Spin Chamber Diameters

Multiple spray tips with differing feeder lengths and spin chamber diameters were analyzed to determine spray coverage properties. Each spray tip had an insert diameter of 3.0 mm, a chamber diameter of 3.3 mm, a chamber height of 6.82 mm, an orifice length of 0.3 mm, and an outlet orifice diameter of 0.26 mm. The varying geometries of each spray tip are detailed in Table 3.

TABLE 3
Spray Tip Geometries

| Design | Feeder Length | Spin Chamber Diameter |
|---|---|---|
| Design A | 0.6 mm | 1.8 mm |
| Design B | 1.15 mm | 0.7 mm |
| Design C | 0.35 mm | 0.6 mm |

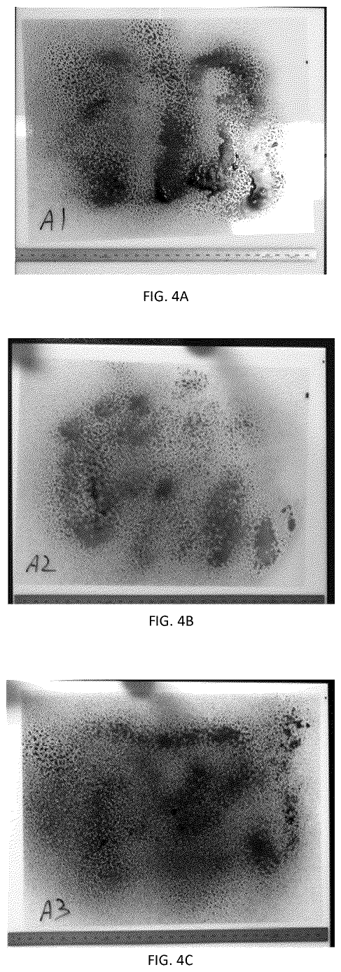

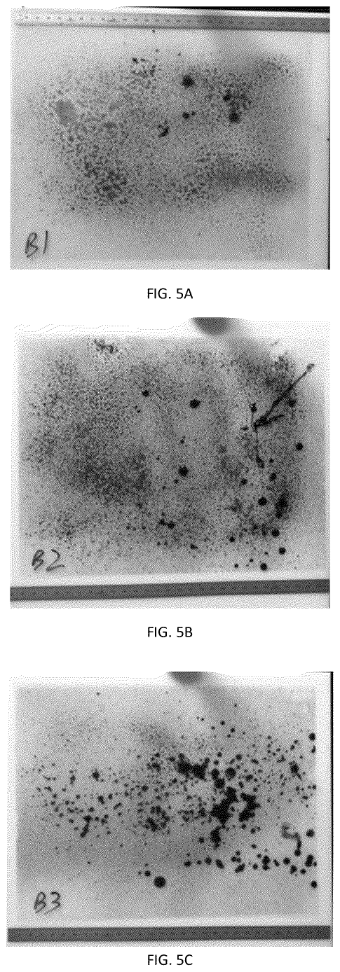

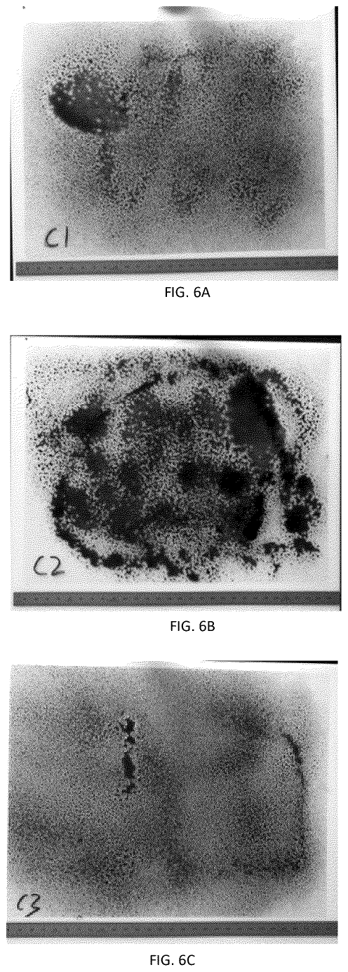

High viscous fluids were prepared with a small amount of gel food coloring and transferred to a syringe. Each spray tip was attached to a fluid syringe for testing. The fluid was sprayed through the spray tip. FIGS. 4A to 4C are images of the spray pattern resulting from Design A. FIGS. 5A to 5C are images of the spray pattern resulting from Design B. FIGS. 6A to 6C are images of the spray pattern resulting from Design C. Table 4 summarizes the results of the test.

TABLE 4
Results of Spray Tip Test

| Sample | Area (cm²) |
|---|---|
| A1 | 281.456 |
| A2 | 300.064 |
| A3 | 438.764 |
| B1 | 234.518 |
| B2 | 341.668 |
| B3 | 163.988 |
| C1 | 371.328 |
| C2 | 374.716 |
| C3 | 428.814 |

Designs A and C exhibited improved spray properties over Design B. A shown in FIGS. 4A to 4C and 6A to 6C, Designs A and C resulted in finer, even spray, while Design B (shown in FIGS. 5A to 5C) produced many globs of material. Feeders shorter in length exhibit improved spray characteristics. When feeders are short, spin chambers with smaller diameters exhibit improved spray characteristics.

### Example 3: Spray Properties of Spray Tips with Different Feeder Sizes

Multiple spray tips with differing orifice diameters, spin chamber lengths, and feeder widths were analyzed to determine spray coverage properties. Each spray tip had a spin chamber diameter of 2.1 mm and 3 feeders. The varying geometries of each spray tip are detailed in Table 5.

TABLE 5
Spray Tip Geometries

| | Orifice | | |
|---|---|---|---|
| | Diameter | Spin Chamber | Feeder Width |
| Sample | (mm) | Length (mm) | (mm) |
| 5A | 0.3 | 1.0 | 0.5 |
| 5B | 0.3 | 1.0 | 0.25 |
| 5C | 0.3 | 0.5 | 0.5 |
| 5D | 0.3 | 0.5 | 0.25 |
| 5E | 0.7 | 2.1 | 0.5 |
| 5F | 0.7 | 2.1 | 0.25 |

High viscous fluids were prepared with a small amount of gel food coloring and transferred to a syringe. Each spray tip was attached to a fluid syringe for testing. The fluid was sprayed through the spray tip. The spray droplet fineness was measured according to metrics in Table 6, with 5 being a desired spray fineness and 0 being an undesirable spray fineness. Spray angle was also measured.

TABLE 6
Spray Droplet Fineness

| | |
|---|---|
| 0 | Stream |
| 1 | Very Course - large single drops |
| 2 | Somewhat Course - several large drops |
| 3 | Neither Course nor Fine - medium sized |
| | drops |
| 4 | Somewhat Fine - many small drops |
| 5 | Very Fine - mist, similar to Spray Set |

The results of the test are shown in Table 7.

TABLE 7
Results of Spray Tip Test

| | Spray Droplet | Time to | Spray Angle | |
|---|---|---|---|---|
| Sample | Fineness | Spray (sec) | (degrees) | Comments |
| 5F | 3 | 3.82 | 30.392 | N/A |
| 5F | 2 | 3.28 | 29.076 | N/A |
| 5A | 0 | 9.68 | 2.090 | N/A |
| 5C | 0 | 10.58 | 3.895 | Stream did not go straight |
| 5B | 4 | 8.59 | 18.246 | Not perfectly circular |
| | | | | spray pattern. Sort of |
| | | | | wide/fan spray. |
| 5B | 4 | 9.83 | 27.808 | Wide/Fan spray pattern |
| 5D | 4 | 9.63 | 54.139 | N/A |
| 5E | 2 | 2.97 | 27.722 | N/A |
| 5A | 0 | 9.66 | 2.271 | Fluctuated between |
| | | | | stream and very narrow |
| | | | | spray |
| 5E | 2 | 2.48 | 51.545. | N/A |
| 5C | 4 | 9.44 | 37.577 | Wide/Fan spray pattern. |
| | | | | Spray angle fluctuates |
| | | | | throughout test. |
| 5D | 4 | 8.60 | 47.181 | Nice circular spray |
| | | | | pattern. Spray angle |
| | | | | fluctuates throughout test |

The results indicate that spray tips with smaller feeder widths (0.25 mm compared to 0.5 mm) exhibit improved spray characteristics for viscous fluids.

The invention is claimed as follows:

1. A spray tip assembly for providing a viscous fluid, the spray tip assembly comprising:
a body comprising an outlet orifice; and
an insert configured to fit within the body,
wherein the spray tip assembly comprises a plurality of channels tangent to and in fluid communication with a hollow chamber, and
wherein each of the plurality of channels has a length between about 0.4 mm and about 1.0 mm.

2. The spray tip assembly of claim 1, wherein a diameter of the hollow chamber is between about 1.6 mm to about 2.2 mm.

3. The spray tip assembly of claim 2, wherein the diameter of the hollow chamber is about 1.8 mm.

4. The spray tip assembly of claim 1, wherein the plurality of channels comprises two channels.

5. The spray tip assembly of claim 1, wherein the outlet orifice is concentric with and smaller than the hollow chamber.

6. The spray tip assembly of claim 1, wherein a width of each of the plurality of channels is between about 0.1 mm and 0.3 mm.

7. The spray tip assembly of claim 6, wherein the width of each of the plurality of channels is about 0.2 mm.

8. The spray tip assembly of claim 1, wherein each of the plurality of channels comprises a square cross-section.

9. The spray tip assembly of claim 1, wherein the body comprises the hollow chamber and the plurality of channels.

10. The spray tip assembly of claim 1, wherein the insert comprises the hollow chamber and the plurality of channels.

11. The spray tip assembly of claim 1, wherein an outside diameter of the body is less than 5.5 mm.

12. The spray tip assembly of claim 1, wherein the body comprises a cylindrical chamber for fitting the insert, and wherein the cylindrical chamber has a diameter of between 3 mm and about 4 mm.

13. A spray tip for mixing and dispensing a viscous fluid, the spray tip comprising:
a swirl chamber at an end portion of the spray tip, the swirl chamber comprising:
a plurality of channels tangent and in fluid communication to a hollow chamber, wherein the length of the plurality of channels is about 0.6 mm or less and the diameter of the hollow chamber is about 1.8 mm or less, and
wherein the swirl chamber is configured to impart rotation to the viscous fluid introduced in the spray tip.

14. The spray tip of claim 13, wherein the length of each of the plurality of channels is 0.6 mm.

15. The spray tip of claim 13, wherein the hollow chamber has a height of between about 0.1 mm to about 0.3 mm.

16. The spray tip of claim 13, wherein the end of the spray tip further comprises an outlet orifice in fluid communication with the hollow chamber, wherein the outlet orifice has a diameter of between about 0.2 mm and 0.3 mm.

17. The spray tip of claim 16, wherein the outlet orifice has a length of between about 0.1 mm and 0.4 mm.

18. The spray tip of claim 13, wherein the spray tip is configured to dispense the viscous fluid at a flow rate of between about 30 ml/min and about 60 ml/min.

19. A spray system comprising:
a multi-component fluid; and
a spray tip for mixing components to form the multi-component fluid and dispensing the multi-component fluid, the spray tip comprising:
a swirl chamber at an end portion of the spray tip, the swirl chamber comprising:
a plurality of channels tangent and in fluid communication to a hollow chamber, wherein the length of the plurality of channels is about 0.6 mm or less and the diameter of the hollow chamber is about 1.8 mm or less, and
wherein the swirl chamber is configured to impart rotation to the multi-component fluid after being mixed within the spray tip.

20. The spray tip system of claim 19, wherein the multi-component fluid comprises a surgical sealant.
